(19) United States
(12) Patent Application Publication
(10) Pub. No.: US 2011/0105884 A1
(43) Pub. Date: May 5, 2011

(54) MRI INVOLVING DYNAMIC PROFILE SHARING SUCH AS KEYHOLE AND MOTION CORRECTION

(75) Inventor: Gabriele Marianne Beck, Eindhoven (NL)

(73) Assignee: KONINKLIJKE PHILIPS ELECTRONICS N.V., EINDHOVEN (NL)

(21) Appl. No.: 12/673,994

(22) PCT Filed: Aug. 20, 2008
(86) PCT No.: PCT/IB08/53326

§ 371 Date: Feb. 18, 2010

(30) Foreign Application Priority Data
Aug. 24, 2007 (EP) 07114923.1

(51) Int. Cl. A61B 5/055; G06K 9/00

(52) U.S. Cl. 600/410; 382/131

(57) ABSTRACT

The invention relates to a device and to a method for magnetic resonance imaging (MRI) of a body. It is an object of the invention to provide a technique that enables dynamic profile sharing with significantly reduced motion artifacts. The method of the (such as keyhole) invention comprises the following steps: a) acquiring an MR data set (21) from an incomplete first part of k-space (C) by subjecting the body to an imaging sequence of RF pulses and switched magnetic field gradients; b) reconstructing an incomplete MR image (31) from the MR data set (21) acquired in step a) and deriving image transformation parameters describing motion of the body from the reconstructed incomplete MR image (31), -c) acquiring an MR data set (24) from an incomplete second part of k-space (P), which second part (P) is different from the first part (C) sampled in step a); d) applying a motion correction (40) to at least one of the MR data sets (21, 24) acquired in steps a) and c) according to the image transformation parameters derived in step b); e) reconstructing a final MR image (81) from a combination (71) of the motion-corrected MR data sets (61, 64).

### FIELD OF THE INVENTION

The invention relates to a device for magnetic resonance (MR) imaging of a body placed in an examination volume.

Furthermore, the invention relates to a method for magnetic resonance imaging (MRI) and to a computer program for an MR device.

### BACKGROUND OF THE INVENTION

In MRI pulse sequences consisting of RF pulses and switched magnetic field gradients are applied to an object (a patient) placed in a homogeneous magnetic field within an examination volume of an MR device. In this way, phase and frequency encoded magnetic resonance signals are generated, which are scanned by means of RF receiving antennas in order to obtain information from the object and to reconstruct images thereof. Since its initial development, the number of clinically relevant fields of application of MRI has grown enormously. MRI can be applied to almost every part of the body, and it can be used to obtain information about a number of important functions of the human body. The pulse sequence, which is applied during an MRI scan, plays a significant role in the determination of the characteristics of the reconstructed image, such as location and orientation in the object, dimensions, resolution, signal-to-noise ratio, contrast, sensitivity for movements, etcetera. An operator of an MR device has to choose the appropriate sequence and has to adjust and optimize its parameters for the respective application.

In a variety of MRI applications, motion of the examined object can adversely affect image quality. Acquisition of sufficient MR data for reconstruction of an image takes a finite period of time. Motion of the object to be imaged during that finite acquisition time typically results in motion artifacts in the reconstructed MR image. In conventional MRI approaches, the acquisition time can be reduced to a very small extent only, when a given resolution of the MR image is specified. In the case of medical MRI, motion artifacts can result for example from cardiac cycling, respiratory cycling, and other physiological processes, as well as from patient motion. In dynamic MRI scans, the motion of the examined object during data acquisition leads to different kinds of blurring, mispositioning and deformation artifacts.

Prospective motion correction techniques such as the so-called navigator technique or PACE have been developed to overcome problems with respect to motion by prospectively adjusting the imaging parameters, which define the location and orientation of the field of view (FOV) etc. within the imaging volume. In the navigator technique hereby, an MR data set is acquired from a pencil-shaped volume (navigator beam) that crosses the diaphragm of the examined patient. The volume is interactively placed in such a way that the position of the diaphragm can be reconstructed from the acquired MR data set and used for motion correction of the FOV in real time. The navigator technique is primarily used for minimizing the effects of breathing motion in cardiac exams. Opposed to the navigator technique, which requires a navigator beam to detect motion differences, the above-mentioned PACE technique uses previously acquired dynamic images to prospectively adjust the imaging parameters on the time scale of successive dynamic scans.

It is further known to diminish the acquisition time in dynamic MRI by sharing the acquired MR data of different sets or scans. This technique is generally referred to as profile sharing. There are different known approaches of profile sharing like the so called “keyhole” method, GES, FAST CARD, TRICKS etc. in which the central region of k-space is acquired more often than the peripheral regions. This is because the examined dynamic processes (for instance in contrast enhanced MRI) affect mainly the MR data acquired from central k-space. For the reconstruction of a time series of high-resolution MR images, multiple MR data sets acquired from central k-space are combined with an MR data set acquired from peripheral k-space. By these techniques, the temporal resolution of dynamic MRI is significantly increased.

The above-described known techniques for dynamic MRI have several drawbacks. Especially in three-dimensional dynamic MRI the time required for acquisition of a single MR data set is still too long to perform an effective motion correction and to avoid motion-induced image artifacts.

### SUMMARY OF THE INVENTION

Therefore, it is readily appreciated that there is a need for an improved MR device. It is consequently an object of the invention to provide an MR device that enables dynamic MRI with significantly reduced motion artifacts.

In accordance with the present invention, a device for magnetic resonance imaging of a body placed in an examination volume is disclosed, which device comprises means for establishing a substantially homogeneous main magnetic field in the examination volume, means for generating switched magnetic field gradients superimposed upon the main magnetic field, means for radiating RF pulses towards the body, control means for controlling the generation of the magnetic field gradients and the RF pulses, means for receiving and sampling magnetic resonance signals, and reconstruction means for forming MR images from the signal samples. In accordance with the invention, the device is arranged, for example by an appropriate programming of the control means and/or the reconstruction means, to

a) acquire an MR data set from an incomplete first part of k-space by subjecting the body to an imaging sequence of RF pulses and switched magnetic field gradients;
b) reconstruct an incomplete MR image from the MR data set acquired in step a) and derive image transformation parameters describing motion of the body from the reconstructed incomplete MR image;
c) acquire an MR data set from an incomplete second part of k-space, which second part is different from the first part sampled in step a);
d) apply a motion correction to at least one of the MR data sets acquired in steps a) and c) according to the image transformation parameters derived in step b);
e) reconstruct a final MR image from a combination of the motion-corrected MR data sets.

The device of the invention is arranged to correct for motion during MR data acquisition using dynamic profile sharing. The technique of the invention is particularly useful in head, feet or leg applications but it can also be used, e.g., for multiple breathhold scans in body applications, especially in contrast enhanced dynamic exams (such as magnetic resonance angiography—MRA) where typically three-dimensional dynamic profile sharing techniques are applied. Such “4D” techniques are made more robust by the approach of the invention.

An insight of the invention is that motion detected from only a part of k-space can be employed to correct the acquired profiles before profile sharing in order to reduce motion artifacts in the finally reconstructed MR images.

In accordance with the invention, an individual incomplete MR image is reconstructed from each MR data set acquired from the first part of k-space. In this context, the term incomplete is to be understood to mean that the MR data set is acquired from a part of k-space which is smaller than the k-space region required to reconstruct the final (complete) MR image from the selected FOV at the selected resolution. After reconstruction of an incomplete MR image, image transformation parameters are derived therefrom. The image transformation parameters describe how the position of the individual pixels (or voxels), image segments or complete image objects (collectively referred to as “image elements”) have changed in the succession of acquired and reconstructed incomplete MR images. Each image element may be assigned one or more image transformation parameters which are derived, e.g., from a comparison of two, preferably temporally successive incomplete MR images, or from a comparison of the respective incomplete MR image with a reference image acquired and reconstructed once at the beginning of the procedure. The derived image transformation parameters may be combined, e.g., in the form of an (affine) image transformation matrix. But it has to be noted that the resulting motion correction operator is not restricted to affine transformation but could include, e.g., a translational and rotational motion of two distinct image elements (such as legs and feet). The motion correction operator is applied to the MR data acquired from the first and/or second part of k-space according to the invention before they are used for profile sharing and for reconstruction of the final MR image. As mentioned before, it is an insight of the invention that motion detected from a part of k-space data can be used for motion correction of k-space data acquired from this part and also from other parts. Finally, the partial k-space data are combined in accordance with the conventional profile sharing approach, and a final MR image is reconstructed from the combined MR data set. This final image does show no or only few motion artifacts. From combinations of several of the (motion corrected) MR data sets acquired from the first part of k-space with a single MR data set acquired from the second part of k-space, a dynamic succession of final MR images can be obtained which are essentially free from motion artifacts.

Preferably, the incomplete first part of k-space sampled in step a) is a central part of k-space and the incomplete second part of k-space sampled in step c) is a peripheral part of k-space. This embodiment of the invention corresponds to the conventional “keyhole” approach with a central ordering of partial k-space acquisitions. From the MR data sets acquired from the central (first) region of k-space, which is acquired more often than the peripheral (second) regions, low-resolution (incomplete) MR images are reconstructed. For the reconstruction of a time series of high-resolution MR images, multiple MR data sets acquired from central k-space are combined with a MR data set acquired from the peripheral k-space region. In accordance with the invention, the image transformation parameters are derived from the low-resolution MR images reconstructed from the central k-space data. These image transformation parameters are used to apply a motion correction to the central and/or peripheral k-space data.

According to a preferred embodiment of the invention, the MR device is arranged to repeat steps a) and b) in order to acquire a plurality of MR data sets from the first part of k-space successively in time, to reconstruct an incomplete MR image from each acquired MR data set immediately after its acquisition, and to derive image transformation parameters from each reconstructed incomplete MR image. On the basis of these image transformation parameters, a motion correction is applied to the imaging parameters of the imaging sequence used in the succession of MR data acquisitions. The imaging parameters, such as, e.g., the strength and directions of the switched magnetic field gradients, determine the location and orientation of the FOV. The motion detected after each acquisition of a partial k-space MR data set is used in accordance with this embodiment of the invention to adapt the acquisition of the subsequent MR data sets from the first and/or second parts of k-space in order to further reduce motion-induced image artifacts.

When deriving image transformation parameters from the acquired MR data in accordance with the invention, it is possible to detect unacceptable motion, meaning that an effective motion correction will not be feasible at all. In this case, the acquired MR data may simply be rejected and the sampling of the respective k-space region may be repeated. Image artifacts due to motion that can not be compensated for are avoided in this way.

The imaging approach of the invention may be combined with different data acquisition and reconstruction techniques. The reconstruction of the partially acquired MR data sets may be performed, for example, according to the well-known POCS or SENSE techniques or other so-called “k-t” type MR data acquisition and reconstruction approaches.

The invention not only relates to a device but also to a method for MR imaging of a body of a patient placed in an examination volume of an MR device. The method comprises the following steps:

a) acquiring an MR data set from an incomplete first part of k-space by subjecting the body to an imaging sequence of RF pulses and switched magnetic field gradients;
b) reconstructing an incomplete MR image from the MR data set acquired in step a) and deriving image transformation parameters describing motion of the body from the reconstructed incomplete MR image;
c) acquiring an MR data set from an incomplete second part of k-space, which second part is different from the first part sampled in step a);
d) applying a motion correction to at least one of the MR data sets acquired in steps a) and c) according to the image transformation parameters derived in step b);
e) reconstructing a final MR image from a combination of the motion-corrected MR data sets.

A computer program adapted for carrying out the imaging procedure of the invention can advantageously be implemented on any common computer hardware, which is presently in clinical use for the control of magnetic resonance scanners. The computer program can be provided on suitable data carriers, such as CD-ROM or diskette. Alternatively, it can also be downloaded by a user from an Internet server.

The enclosed drawings disclose preferred embodiments of the present invention. It should be understood, however, that the drawings are designed for the purpose of illustration only and not as a definition of the limits of the invention.

### BRIEF DESCRIPTION OF THE DRAWINGS

In the drawings:

FIG. 1 shows an MR scanner according to the invention;

FIG. 2 illustrates the dynamic profile sharing technique of the invention as a flow chart.

### DETAILED DESCRIPTION OF THE EMBODIMENTS

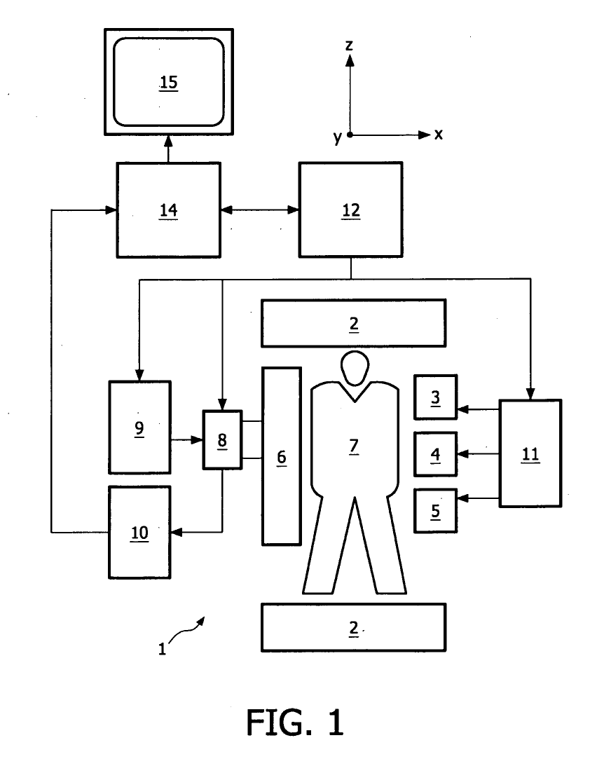

In FIG. 1 an MR imaging device 1 in accordance with the present invention is shown as a block diagram. The apparatus 1 comprises a set of main magnetic coils 2 for generating a stationary and homogeneous main magnetic field and three sets of gradient coils 3, 4 and 5 for superimposing additional magnetic fields with controllable strength and having a gradient in a selected direction. Conventionally, the direction of the main magnetic field is labeled the z-direction, the two directions perpendicular thereto the x- and y-directions. The gradient coils 3, 4 and 5 are energized via a power supply 11. The imaging device 1 further comprises an RF transmit antenna 6 for emitting radio frequency (RF) pulses to a body 7. The antenna 6 is coupled to a modulator 9 for generating and modulating the RF pulses. Also provided is a receiver for receiving the MR signals, the receiver can be identical to the transmit antenna 6 or be separate. If the transmit antenna 6 and receiver are physically the same antenna as shown in FIG. 1, a send-receive switch 8 is arranged to separate the received signals from the pulses to be emitted. The received MR signals are input to a demodulator 10. The send-receive switch 8, the modulator 9, and the power supply 11 for the gradient coils 3, 4 and 5 are controlled by a control system 12. Control system 12 controls the phases and amplitudes of the RF signals fed to the antenna 6. The control system 12 is usually a microcomputer with a memory and a program control. The demodulator 10 is coupled to reconstruction means 14, for example a computer, for transformation of the received signals into images that can be made visible, for example, on a visual display unit 15. For the practical implementation of the invention, the control system 12 and the reconstruction means 14 comprise a programming for carrying out the imaging procedure of the invention.

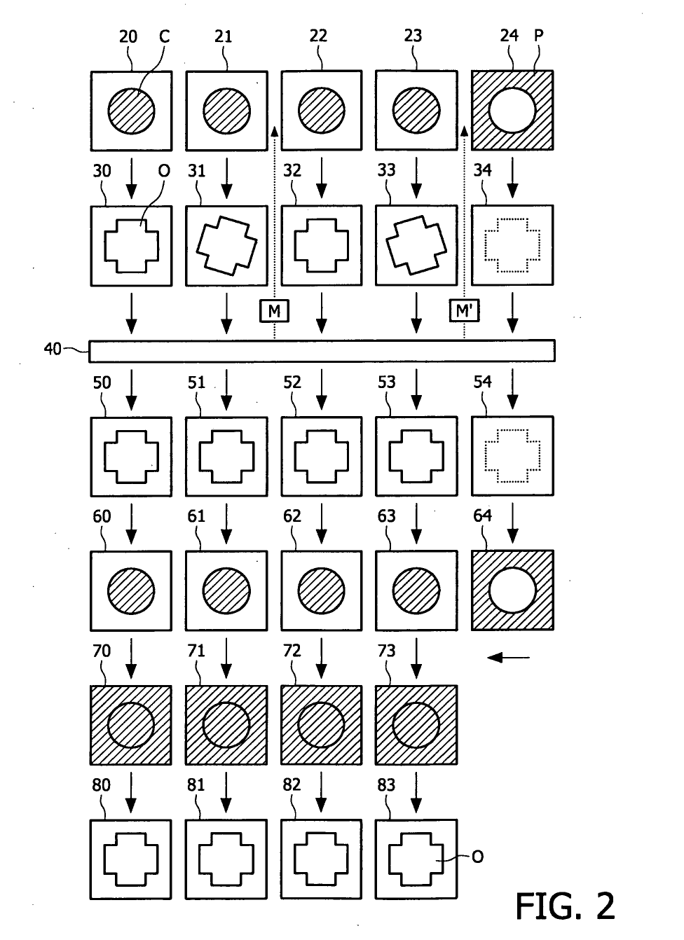

FIG. 2 illustrates the dynamic profile sharing technique of the invention as a flow chart. The imaging procedure starts with the acquisition of an MR data set 20 from an incomplete first part of k-space, namely from a central k-space region C. A standard gradient echo imaging sequence may be used for this purpose. Immediately after data acquisition, a low-resolution MR image 30 is reconstructed from MR data set 20 by inverse Fourier transformation. The low-resolution MR image 30 constitutes an incomplete MR image within the meaning of the invention because the central k-space region C is smaller than the k-space region required to reconstruct an MR image at the full resolution. MR image 30 shows the examined object O in the centre of the FOV. MR image 30 is taken as a reference image during the further imaging procedure. As a next step, a successive MR data set 21 is acquired from central k-space region C and another low-resolution MR image 31 is reconstructed therefrom. MR image 31 shows object O having changed its position and orientation because of motion. In step 40, a comparison of reference image 30 and low-resolution MR image 31 takes place in order to derive image transformation parameters. These image transformation parameters describe how the position of object O has changed in the succession of low-resolution MR images 30 and 31. The image transformation parameters are combined in the form of an affine image transformation matrix. The corresponding matrix operator M is then used to perform a motion correction of the imaging parameters of the imaging sequence applied in the acquisition of subsequent MR data set 22. The imaging parameters, such as, e.g., the strength and directions of the switched magnetic field gradients, determine the location and orientation of the FOV. The FOV is adapted such that the detected motion of the object O is compensated for. The same procedure is repeated for the acquisition of MR data sets 22 and 23 from central k-space. Matrix operator M′ derived from a comparison of reference 30 and low-resolution MR image 33 is used to adapt the imaging parameters for the acquisition of an MR data set 24 from peripheral k-space region P. The peripheral k-space region P constitutes an incomplete second part of k-space within the meaning of the invention. An MR image 34 is reconstructed from MR data set 34 again by inverse Fourier transformation. The image transformation matrices derived from MR images 30, 31, 32, 33, and 34 are used in a post-processing step to compute corresponding motion corrected MR images 50, 51, 52, 53, and 54. As further illustrated in FIG. 2, motion corrected MR images 50, 51, 52, 53, and 54 all show object O in the center of the FOV. A Fourier transform is applied to each MR image 50, 51, 52, 53, and 54 in order to obtain a succession of motion corrected MR data sets 60, 61, 62, and 63 from central k-space and a motion corrected MR data set 64 from peripheral k-space. Finally, combinations 70, 71, 72, and 73 of central k-space MR data sets 60, 61, 62, and 63 with peripheral MR data set 64 are computed and a dynamic succession of high-resolution final MR images 80, 81, 82, and 83 is reconstructed by a further inverse Fourier transformation. High-resolution MR images 80, 81, 82, and 83 are essentially free from motion artifacts.

1. Device for magnetic resonance imaging of a body (7) placed in an examination volume, the device (1) comprising:
means (2) for establishing a substantially homogeneous main magnetic field in the examination volume;
means (3, 4, 5) for generating switched magnetic field gradients superimposed upon the main magnetic field;
means (6) for radiating RF pulses towards the body (7;
control means (12) for controlling the generation of the magnetic field gradients and the RF pulses;
means (10) for receiving and sampling magnetic resonance signals; and
reconstruction means (14) for forming MR images from the signal samples; the device (1) being arranged to:
a) acquire an MR data set from an incomplete first part of k-space by subjecting the body (7) to an imaging sequence of RF pulses and switched magnetic field gradients;
b) reconstruct an incomplete MR image from the MR data set acquired in step a) and derive image transformation parameters describing motion of the body (7) from the reconstructed incomplete MR image;
c) acquire an MR data set from an incomplete second part of k-space, which second part is different from the first part sampled in step a);
d) apply a motion correction to at least one of the MR data sets acquired in steps a) and c) according to the image transformation parameters derived in step b);
e) reconstruct a final MR image from a combination of the motion-corrected MR data sets.

2. Device of claim 1, wherein the incomplete first part of k-space sampled in step a) is a central part of k-space and wherein the incomplete second part of k-space sampled in step c) is a peripheral part of k-space.

3. Device of claim 1 or 2, wherein the device (1) is further arranged to:
repeat steps a) and b) in order to acquire a plurality of MR data sets from the first part of k-space successively in time;
reconstruct an incomplete MR image from each partially acquired MR data set immediately after its acquisition; and
derive image transformation parameters from each reconstructed incomplete MR image.

4. Device of any one of claims 1-3, wherein the device (1) is further arranged to use the image transformation parameters derived from an incomplete MR image to apply a motion correction to the imaging parameters of the imaging sequence used for the acquisition of a subsequent MR data set.

5. Method for MR imaging of a body of a patient placed in an examination volume of an MR device, the method comprising the following steps:
a) acquiring an MR data set (21) from an incomplete first part of k-space (C) by subjecting the body to an imaging sequence of RF pulses and switched magnetic field gradients;
b) reconstructing an incomplete MR image (31) from the MR data set (21) acquired in step a) and deriving image transformation parameters describing motion of the body from the reconstructed incomplete MR image (31);
c) acquiring an MR data set (24) from an incomplete second part of k-space (P), which second part (P) is different from the first part (C) sampled in step a);
d) applying a motion correction (40) to at least one of the MR data sets (21, 24) acquired in steps a) and c) according to the image transformation parameters derived in step b);
e) reconstructing a final MR image (81) from a combination (71) of the motion-corrected MR data sets (61, 64).

6. Method of claim 5, wherein the incomplete first part of k-space (C) sampled in step a) is a central part of k-space and wherein the incomplete second part of k-space (P) sampled in step c) is a peripheral part of k-space.

7. Method of claim 5 or 6, wherein steps a) and b) are repeated in order to acquire a plurality of MR data sets (20, 21, 22, 23) from the first part of k-space (C) successively in time, an incomplete MR image (30, 31, 32, 33) being reconstructed from each acquired MR data set (20, 21, 22, 23) immediately after its acquisition, and wherein image transformation parameters are derived from each reconstructed incomplete MR image (30, 31, 32, 33).

8. Method of any one of claims 5-7, wherein the image transformation parameters derived from an incomplete MR image (30, 31, 32, 33) are used to apply a motion correction to the imaging parameters of the imaging sequence used for the acquisition of a subsequent MR data set (20, 21, 22, 23, 24).

9. Method of any one of claims 5-8, wherein a dynamic succession of final MR images (80, 81, 82, 83) is reconstructed from combinations (70, 71, 72, 73) of a single MR data set (64) acquired from the second part of k-space (P) with different motion corrected MR data sets (60, 61, 62, 63) successively acquired from the first part (C) of k-space.

10. Computer program for an MR device, comprising instructions for:
a) acquiring an MR data set from an incomplete first part of k-space by generating an imaging sequence of RF pulses and switched magnetic field gradients;
b) reconstructing an incomplete MR image from the MR data set acquired in step a) and deriving image transformation parameters describing motion of an examined object from the reconstructed incomplete MR image;
c) acquiring an MR data set from an incomplete second part of k-space, which second part is different from the first part sampled in step a);
d) applying a motion correction to at least one of the MR data sets acquired in steps a) and c) according to the image transformation parameters derived in step b);
e) reconstructing a final MR image from a combination of the motion-corrected MR data sets.

11. Computer program of claim 10 comprising further instructions for:
repeating steps a) and b) in order to acquire a plurality of MR data sets from the first part of k-space successively in time,
reconstructing an incomplete MR image from each acquired MR data set immediately after its acquisition, image transformation parameters being derived from each reconstructed incomplete MR image in order to apply a motion correction to the imaging parameters of the imaging sequence used for the acquisition of the subsequent MR data set,
reconstructing a dynamic succession of final MR images from combinations of a single MR data set acquired from the second part of k-space with different motion corrected MR data sets successively acquired from the first part of k-space.
